(19) United States
(12) Patent Application Publication
(10) Pub. No.: US 2022/0313076 A1
(43) Pub. Date: Oct. 6, 2022

(54) EYE VISION TEST HEADSET SYSTEMS AND METHODS

(71) Applicant: VR EYE TEST, LLC, Aliso Viejo, CA (US)

(72) Inventor: Omar Krad, Aliso Viejo, CA (US)

(73) Assignee: VR EYE TEST, LLC, Aliso Viejo, CA (US)

(21) Appl. No.: 17/219,304

(22) Filed: Mar. 31, 2021

(51) Int. Cl. A61B 3/024; A61B 3/00; G02B 27/01

(52) U.S. Cl. CPC A61B 3/024; A61B 3/0033; G02B 27/0172; A61B 3/0041

(57) ABSTRACT

A system for conducting eye vision tests includes a patient-wearable headset having first and second displays configured to be visible separately to each of the patient's eyes, and a user interface that allows a user to provide vision test feedback. Visual field tests are conducted using the headset to determine a patient's visual field zone, contrast sensitivity, and reaction times, thereby establishing a calibration customized to each patient. The system is operable to perform tests of visual conditions, such as torsion and strabismus, for each of the patient's eyes.

### CROSS-REFERENCE TO RELATED APPLICATIONS

None

### FEDERALLY SPONSORED RESEARCH OR DEVELOPMENT

Not Applicable

### BACKGROUND

Vision tests traditionally must be conducted within an optometrist's office using specialized equipment, such as phoropter machines or digital refraction machines. Conducting such tests in the office, however, requires a patient to travel to an eye care provider (e.g., doctor, optometrist, optician) which is not always convenient, and such machines can be expensive to procure and maintain.

It would therefore be desirable to have improved, portable vision tests that can be conducted in a less expensive manner in locations remote from an eye care provider.

### SUMMARY

Systems and methods for conducting vision tests using a headset are disclosed. The headset comprises a first display or screen configured to be positioned in front of one eye of a patient, and a second display or screen configured to be positioned in front of the other eye of a patient. The headset is preferably coupled to a user interface that allows a patient to provide inputs to the headset, for example inputs that allow a user to indicate when objects are viewed on one display or screen or the other, and to indicate if a first displayed object is aligned with a second displayed object on the same display or screen, or on the other display or screen. (For the purpose of this disclosure, the terms “screen” and “display” encompass any suitable type of visual display.)

Various calibration methods could be used to determine a patient's contrast sensitivity, reaction times, and blind spot locations. For example, to determine a patient's blind spot locations, a headset could be configured to display a focus point on a display and provide an instruction to the patient to focus on the focus point. The headset could then display calibration points on various locations on the display, soliciting feedback from the patient on whether the patient can see a calibration point in a location while the patient is focusing on the focus point. As the system records various calibration points in various locations on the screen, the system can determine a visual zone of areas that the patient can see, and blind spot locations which the patient cannot see. Once a visual zone has been established for a patient, the headset could save and record that visual zone in a database location specific to that patient, and/or to a unique identifier of the patient, which allows the headset to conduct tests in the future using that visual zone without needing to recalibrate the headset every time. A different calibration test and a different visual zone can be generated independently in each screen for each eye, allowing for different visual zones to be established for each eye.

Other methods could be utilized to calculate a reaction time of a patient. For example, a headset system could transmit a test point for display on a screen, preferably within a visual zone of a patient for that eye. The system could then receive a signal from the user interface that the patient sees the first test point on the screen, and the headset could then record the time delay between the transmission of the first test point and the reception of the signal from the patient from the user interface that the patient sees the first test point. By conducting this calibration method several times within the visual zone of the patient, the system could calculate minimum, maximum, mean, and median reaction times for the patient, which could be advantageously utilized in further tests. In some embodiments, the headset system could generate a maximum reflex time that is greater than any of the recorded time delays between transmission of a test point and reception of a signal from the patient that the patient sees the test point. Such tests could also be conducted with each eye independently, and with different user interfaces, such as a user interface for each hand of a patient or a user interface for the voice of a patient. Conducting such tests with different user interface inputs independently from one another allows reaction times for different input modes to be calculated as well, as a user's left and right hands may have different reaction times. The system could then use the maximum reflex time as a threshold time period to wait for a signal from the patient before displaying another test point for an eye exam test.

Other methods could be utilized to calculate perceived contrast levels for a patient, which could be utilized to simulate brightness. For example, a headset system could transmit calibration points with differing luminance values from one another, or differing luminance values from a background image that the calibration points are displayed upon, while providing instructions to the patient to indicate whether a calibration point is seen on the screen, and/or whether a patient perceives a calibration point to be too bright or painful for the patient. Luminance differences between calibration points that are indicated to be seen by a patient, and not seen by a patient, could be recorded, and used to determine minimum luminance differences that can be seen, and maximum luminance differences that are perceived to be painful to the patient. In some embodiments, a maximum or a minimum luminance value of a pixel could be set by the system as a function of the maximum or minimum brightness thresholds provided by user feedback. For example, a user could indicate to the system that a brightness of greater than 200 of an RGB (Red-Green-Blue) value (where the value of each of R, G, and B is set between 0 and 255) is too bright to tolerate, whereas a value below 50 is too dark to differentiate from a pure black background having values of 0-0-0. With such feedback, the system could designate thresholds that brighten a pixel to have values greater than 50-50-50 and less than 200-200-200 for that user, automatically dimming any pixel having a value greater than 200 to be set at the 200 maximum, or automatically brightening any pixel having a value less than 50 to be set at the 50 minimum.

Generated visual zones, maximum reaction times, and minimum/maximum luminance values for a patient could be saved to a database location that is keyed to a patient, or to a unique identifier of the patient, to allow visual tests to be provided to a patient using the headset repeatedly without needing to recalibrate the system every time. In some embodiments, a patient could be given a unique identifier, such as a barcode or a number, that could be input into a headset system during calibration, and before tests are performed, to allow a patient to associate a calibration with the unique identifier, and to load such a calibration before tests are performed. In some embodiments, a patient could be prompted to perform a calibration after threshold time periods have passed, such as six months or a year, or when an administrator user, such as a doctor, a nurse, or eye care practitioner, transmits a notification to a system that the patient should recalibrate. By saving a calibration to a commonly accessed database, a user could use different headsets with the same calibration without needing to perform a calibration test again.

Various contemplated tests could be performed using a visual headset in accordance with this disclosure, such as stereo testing, visual acuity testing, and strabismus and torsion testing. For example, in an embodiment where a headset is used to perform stereo testing, a stereo depth-perception test could be provided to one or both screens of a headset to identify vision problems and conduct a graded circle test to measure a patient's depth perception. In such embodiments, a patient could be presented with multiple circles that contain a dot within a circle, and be provided an instruction to indicate which dot “pops out” of the plane of the circle and appears to be 3D according to the patient's vision. A user interface, such as a mouse or a touch controller, could be used to allow a user to indicate which circle is indicated. In some embodiments, separate graded circle tests could be presented to each eye, and/or the same graded circle test could be presented to both eyes. Similar methods could be utilized to perform visual acuity testing, where the system presents a Snellen eye chart, and instructs a patient to read letters and numbers, or select all letters or numbers of a certain type on the chart. For example, a user could be instructed to select all U's seen on a chart using a user interface, or a user could be instructed to select all circles with bullseyes in a chart.

In an embodiment where a headset is configured to perform a strabismus measurement test, a headset system could be configured to display a test point on each screen of a headset within the user's visual zone. Preferably, each test point is displayed at the same coordinates for each screen, for example the center of each screen. The system could then solicit feedback from the patient to determine what the patient sees. A patient who indicates to the system that the patient only sees one point may have perfect vision, but a patient who indicates to the system that the patient sees two different points may have a strabismus issue. The severity of the strabismus could be measured by allowing a user to move a point on a display from one location to another until, to the user, both points align with one another. Each point could be colored differently to allow for easy differentiation between the points. Each point could be moved independently to allow measurements of each eye's strabismus. The horizontal and vertical deviations could be measured and used to calculate the patient's strabismus severity, and the system could save historical test results to allow a patient or an eye care practitioner to see how a strabismus may change over time.

In an embodiment where a headset is configured to perform a torsion measurement test, a headset system could be configured to display a test line, such as a horizontal line or a vertical line, on each screen of a headset within the user's visual zone. Similar to the strabismus test, the lines are preferably displayed in the same location for each screen having the same coordinates and the same angle of rotation. The system could then solicit feedback from the patient to determine what the patient sees. A patient who indicates to the system that the patient only sees one line may have perfect vision, but a patient who indicates to the system that the patient sees two different lines may have a torsion issue. The severity of the torsion could be measured by allowing a user to move and rotate a line from one location on a screen to another until, to the user, both lines align with one another. Each line could be colored differently to allow for easy differentiation between the lines. Each line could also be moved independently to allow measurements of each eye's torsion. The angle of rotation until the lines are aligned with one another could be measured and used to calculate the patient's torsion severity, and the system could save historical test results to allow a patient or a practitioner to see how a torsion may change over time. In some embodiments, a torsion test and a strabismus test may be combined, as the horizontal and vertical deviation could also be calculated by varying the thickness of a line on a screen.

A headset system could be configured to provide instructions via a virtual assistant, which provides step by step instructions on various functions of the headset system, such as how to take a test, what information to provide, or how to provide information. The virtual assistant could provide instructions in any suitable manner, for example by providing text on a screen, by providing audio instructions, or by providing a visual representation of an eye care practitioner that provides instructions to a user of the headset system. In some embodiments, the virtual assistant could visually display instructions as text on a screen that act as a focus point for the user to look at. In preferred embodiments, the virtual assistant provides an audio component that allows a user to listen to instructions while looking at a focal point, thereby allowing a smaller focal point to be the area of focus for a user. In some embodiments, the headset system could present a user with a visual representation of a practitioner's office, allowing a user to look at a visual representation of a wall or a display screen in the office that could act as the platform for a test.

The virtual assistant is preferably configured to provide real-time feedback to a user patient who responds to a visual cue or an audio signal by actuating a response switch that may be configured, for example, as a button or a trigger. For example, if the headset system detects that a user patient actuates the response switch after the passage of a period of time greater than the user's known maximum reaction time, the virtual assistant could provide visual or audio feedback to the user patient that the patient is not actuating the response switch fast enough. In another embodiment, the headset system could detect that a user patient actuates a response switch after a light is presented within a visual blind spot area, which indicates that the user is not looking at the focus point. When the system detects such feedback, the virtual assistant could be configured to remind the patient to look at the focal point. In this manner, instructions that are provided to a user patient could be provided via an intuitive virtual assistant that provides real-time feedback via the user interface. In other embodiments, the system could be configured to visually present an FAQ menu, portions of which could be selected to activate a three-dimensional stereo video of a practitioner that answers a question using pre-recorded footage, which simulates an in-office experience.

In some embodiments, the tests that are provided to a patient are selected by an eye care practitioner and are triggered by an action by a patient, for example by inputting a patient UID or by scanning a QR code into the headset system. In other embodiments, the tests that are provided to a patient are selected by the patient when the patient is engaged with the system. Other variations on the disclosed embodiments are envisioned, as explained in the detailed description below.

### BRIEF DESCRIPTION OF THE DRAWINGS

FIG. 1 shows a schematic of an exemplary portable headset coupled with a network system to transmit data between one or more servers and client computer systems.

FIG. 2 shows a logical schematic of an exemplary headset system in accordance with this disclosure.

FIG. 3 shows a first display or screen and a second display or screen used to perform calibration and vision testing for a patient.

FIG. 4A shows an exemplary method for calibrating a visual zone of a patient.

FIG. 4B shows an exemplary method for verifying a visual zone of a patient.

FIG. 4C shows an exemplary method for calibrating a virtual brightness threshold of a patient.

FIG. 5 shows a first display or screen and a second display or screen used to perform a strabismus measurement for a patient.

FIG. 6 shows a first display or screen and a second display or screen used to perform a torsion measurement for a patient.

FIG. 7 shows a first display or screen and a second display or screen used to perform a combined strabismus and torsion measurement for a patient.

FIG. 8A-8C show displays or screens configured to provide different types of virtual assistants when conducting tests for a patient.

### DETAILED DESCRIPTION

The following detailed description describes various headset embodiments that are designed to calibrate and perform visual tests for a patient.

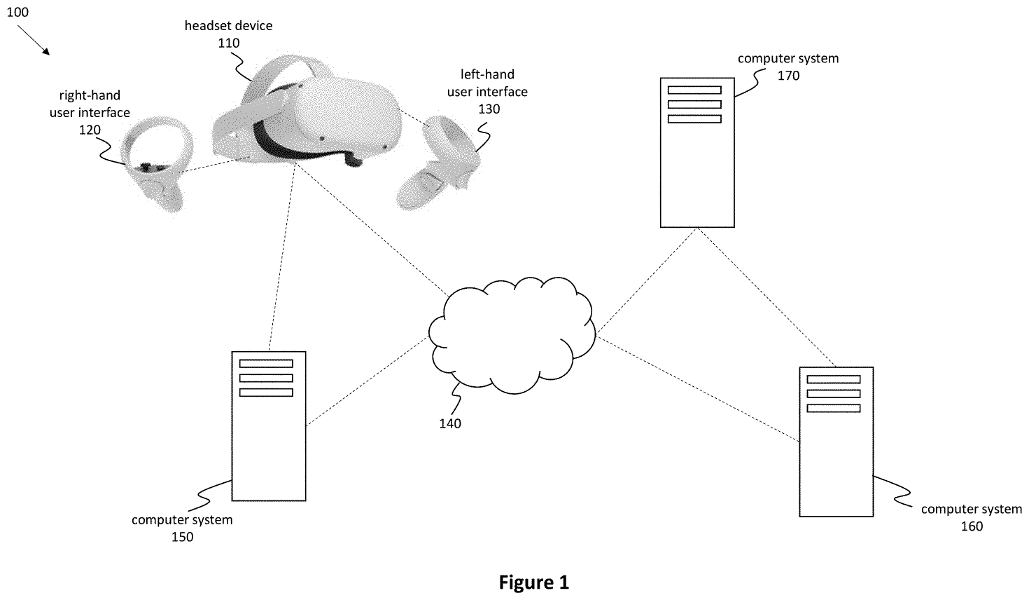

FIG. 1 illustrates a schematic 100 of an exemplary headset 110 having a right-hand user interface 120 and a left-hand user interface 130 functionally coupled to one or more computer systems 150, 160, 170, and 180 using a network 140. The headset 110, as shown, may be configured to worn on a patient's head, with a pair of screens, one for each eye of the patient. In some embodiments, the headset 110 could be a virtual reality headset that allows a computer system with a screen, preferably a portable computer system, such as a tablet or a cellphone (not shown), to be placed in front of a user's face with dividers that block one part of the screen from being seen by the left eye of the wearer and another part of the screen from being seen by the right eye of the wearer, allowing for a single screen to be used to show two separate displays independently from one another to a wearer of the headset. In some embodiments, the headset 110 may also have a microphone (not shown) and/or a speaker (not shown) that could be used, respectively, to transmit audio instructions to a wearer, and to receive audio input from a wearer. In such embodiments, a headset could comprise one or more channels that are configured to direct audio sound to the microphone and from the speakers of the computer system. In other embodiments, the headset 110 could have an embedded computer system (not shown) built into the headset, having custom-built screens, microphones, and/or speakers built into the headset computer system to allow the headset to conduct tests without needing other computer systems to be connected (wired or wirelessly) to the headset.

As used herein, a “computer system” comprises any suitable combination of computing or computer devices, such as desktops, laptops, cellular phones, blades, servers, interfaces, systems, databases, agents, peers, engines, modules, or controllers, operating individually or collectively. Computer systems and servers may comprise at least a processor configured to execute software instructions stored on a tangible, non-transitory computer readable storage medium (e.g., hard drive, solid state drive, RAM, flash, ROM, etc.). The software instructions preferably configure the computer system and server to execute the functionality as disclosed.

User interfaces 120 and 130 are shown as touch controllers of the type having accelerometers (not shown), and that allow a connected computer system, such as a computer system embedded in the headset 110, to communicate with the user interfaces 120 and 130 and receive input from a user. While the user interfaces 120 and 130 are shown as touch controllers having triggers and accelerometers to detect movement of the controllers in X-Y-Z directions, any suitable user interfaces could be used to transmit data to a headset computer system, such as a user-actuatable switch or button in a mouse or a keyboard. In other embodiments, a user interface could be embedded and/or incorporated within the headset 110 itself, such as an accelerometer that detects movement of the patient's head, or a microphone that accepts audio input from a patient. The user interfaces could be functionally connected to the headset computer system in any suitable manner, such as wired or wireless connections like a Bluetooth® or WiFi connection.

The headset 110 is advantageously configured by one or more computer systems 150, 160, and 170 to transmit data to and from the headset 110. Such data could include any suitable data used by the disclosed systems, such as configuration data, calibration data, and test data. The computer system 150, for example, could be a patient's computer system utilized to store data specific to a patient, while a server computer system 160 could be utilized to store data for a plurality of patients. The patient computer system 150 could be functionally connected to the computer system in the headset 110 via a wired or wireless connection, or it could be functionally connected to the computer system in the portable headset 110 via the network 140. As used herein, a “network” comprises to any type of data, telecommunications or other network including, without limitation, data networks (including MANs, PANs, WANs, LANs, WLANs, micronets, piconets, internets, and intranets), hybrid fiber coax (HFC) networks, satellite networks, cellular networks, and telco networks. Such networks or portions thereof may utilize any one or more different topologies (e.g., ring, bus, star, loop, etc.), transmission media, and/or communications or networking protocols and standards (e.g., SONET, DOCSIS, IEEE Std. WAP, FTP).

The computer system 170 may be a practitioner (physician, optometrist, or other eye care practitioner) computer system, which would functionally couple, either directly or indirectly, to the server computer system 160 to retrieve data on any patients who have uploaded their data to the server computer system 160 during use. In preferred embodiments, a patient who utilizes a headset 110 could be given a unique identifier, such as a barcode or a number, that could be input into a headset system using a user interface, such as the user interfaces 120 or 130. Such a unique identifier could be used to upload patient data to the server computer system 160 to save data, such as calibration information and/or test information, and to allow a patient or practitioner to retrieve such saved information from the server as needed using the unique identifier. In some embodiments, a patient could be prompted to perform a calibration after threshold time periods have passed, such as six months or a year, or when an administrator user, such as an eye care practitioner, transmits a notification to a system that the patient should recalibrate. By saving a calibration to a commonly accessed database, a user could use different headsets with the same calibration without needing to perform a calibration test again.

The headset 110 may be configured to allow a patient user to create their own user profile, enter in profile-specific information (e.g., name, date of birth, email address, whether they are wearing glasses or contact lenses), and select from an assortment of vision tests listed on a menu. Such entered profile information and test result data could be saved to a database on any suitable computer system accessible to the headset 110, such as a memory on the headset 110, a commonly accessed database saved on a remote server 160, or a locally accessed database saved on local patient computer system 150.

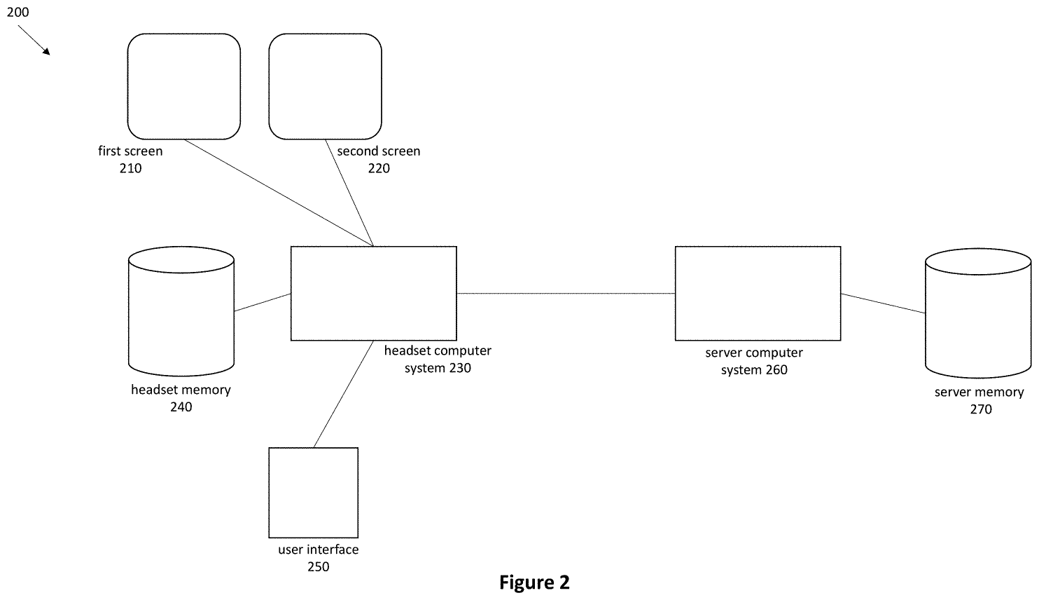

FIG. 2 shows a logical schematic of an exemplary portable headset system 200 having a left screen display 210, a right screen display 220, a headset computer system 230, a headset memory 240, at least one user interface 250, a server computer system 260, and a server memory 270. The headset computer system 230 has a processor that executes instructions saved on the headset memory 240, transmits data to display on the displays 210 and 220, and receives inputs from the one or more user interfaces 250. The headset computer system 230 could also be configured to communicate with a server 260 having a memory 270 that saves patient data from one or more patients.

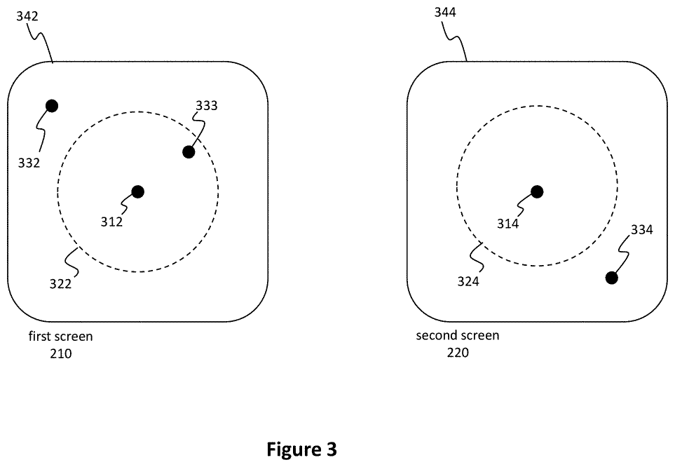

FIG. 3 shows a first screen 210 and a second screen 220 used to perform calibration and vision testing for a patient. When a headset computer system performs calibration of a patient's blind spot locations, a headset could be configured to display a first focus point 312 on the first screen 210 and provide an instruction to the patient to focus on the first focus point 312 on the first screen 210. Such instructions could be provided in any suitable manner, for example via a banner that displays on the screen, or an audio instruction that is transmitted by a speaker of the headset computer system. The headset computer system could then display calibration points on various locations on the screen, such as first screen calibration points 332 and 333, soliciting feedback from the patient on whether the patient can see a calibration point in a location while the patient is focusing on the first focus point 312. In some embodiments, the system could display additional calibration points on the screen. As the system records which calibration point locations a patient can and cannot see while focusing on the first focus point 312, the system can determine a first visual zone 322 of areas that the patient can see with one eye, and a first blind spot zone 342 which the patient cannot see with that eye. In some embodiments, multiple visual zones or multiple blind spot zones could be created to create a patchwork of zones that could be utilized by the headset computer system. Once a first visual zone 322 has been established for a patient, the headset computer system could save and record that first visual zone 322 in a database specific for that patient, or a unique identifier of the patient, which allows the headset to conduct tests in the future within that visual zone without needing to recalibrate the headset every time.

As shown, a different calibration test and a different visual zone can be conducted independently from each screen for each eye, allowing for different visual zones to be established for each eye. Here, the second screen 220 has a second focus point 314 and at least one second screen calibration point 334 that is shown to determine a second visual zone 324 and a second blind spot zone 344 of the patient's other eye. In some embodiments, the calibration tests for each eye can be performed sequentially or interleaved with one another. For example, in some embodiments the headset computer system could perform a calibration test for a patient's left eye, then a patient's right eye, or it could show a calibration point for the left eye first, then the right eye, and then the left eye again, and so on. In other embodiments, the headset computer system could conduct calibration tests for both eyes simultaneously, displaying a calibration point in the same coordinates for the left eye display 210 as for the right eye display 220, which allows a system to determine if a different visual zone might need to be created for embodiments where images are transmitted to both eyes simultaneously. In other embodiments, the headset computer system could be configured to generate a “both eye visual zone” by including the visual zones for both the left eye calibration test and the right eye calibration test.

Other methods could be utilized to calculate a reaction time of a patient. For example, a headset system could instruct a patient to transmit a signal indicating that the patient sees a calibration point on a screen of the headset. The signal may be transmitted by actuating a switch, such as, for example, by pulling a trigger of a touch controller user interface or by clicking a mouse button user interface. The headset system could then transmit a first calibration point 332 for display on the first screen 210, preferably within the first visual zone 322, and receive a signal from the user interface that the patient sees the first calibration point 332 on the first screen 210. The headset computer system could then record the time delay between the transmission of the first calibration point 322 to the first display 210 and the reception of the signal from the patient from the user interface that the patient sees the first calibration point. By conducting this calibration method several times within the first visual zone 322 of the patient, the system could calculate minimum, maximum, mean, and median reaction times for the patient, which could be advantageously utilized in further tests. For example, the headset system could be configured to ensure that all tests are conducted such that a delay between displayed content must be above the maximum reaction time of the patient to ensure that the system records all reactions from the patient, or the headset system could be configured to ignore inputs that are received below a minimum reaction time for a patient between the time a calibration point is displayed and a signal is received from the user interface. In some embodiments, the headset system could generate a maximum reflex time that is greater than any of the recorded time delays between transmission of a calibration point and reception of a signal from the patient.

As before, such tests could also be conducted with each eye independently by performing the calibration test on each screen 210 and 220 independently. In some embodiments, the reaction time calibration test could be conducted with different user interfaces independently, such as a user interface for each hand of a patient or a user interface for the voice of a patient. In such a manner, the calculated reaction time for a patient's left hand may be a different value than the calculated reaction time for a patient's right hand. Conducting such tests with different user interface inputs independently from one another allows reaction times for different input modes to be calculated as well, since a user's left and right hands may have different reaction times.

The headset system could also transmit each calibration point 332, 333, and 334 with differing luminance values, or differing luminance values from a background image that the calibration points are displayed upon. For example, a background image could have a luminance value of 4 while a calibration point has a luminance value of 8, or a background image could have a luminance value of 12 while a calibration point has a luminance value of 3. The headset system could also provide instructions to the patient to indicate whether a calibration point is seen on the screen, and/or whether a patient perceives a calibration point to be too bright or too painful for the patient. Luminance differences between calibration points that are indicated to be seen by a patient, and not seen by a patient, could be recorded, and used to determine minimum luminance differences that can be seen, and maximum luminance differences that are perceived to be painful to the patient. Such luminance differences could then be used in further tests. For example, the headset system could calculate minimum and maximum luminance difference to be used for various tests for a patient, to ensure that a patient can see test images without pain or discomfort.

Generated visual zones, maximum reaction times, and minimum/maximum luminance values for a patient could be saved to a database that is keyed to a patient, or to a unique identifier of the patient, to allow visual tests to be provided to a patient using the headset repeatedly without needing to recalibrate the system every time. In some embodiments, a patient could be given a unique identifier, such as a barcode or a number, that could be input into a headset system during calibration, and before tests are performed, to allow a patient to associate a calibration with the unique identifier, and load such a calibration before tests are performed. In some embodiments, a patient could be prompted to perform a calibration after threshold time periods have passed, such as six months or a year, or when an administrator user, such as an eye care practitioner, transmits a notification to a system that the patient should recalibrate. By saving a calibration to a commonly accessed database, a user could use different headsets with the same calibration without needing to perform a calibration test again.

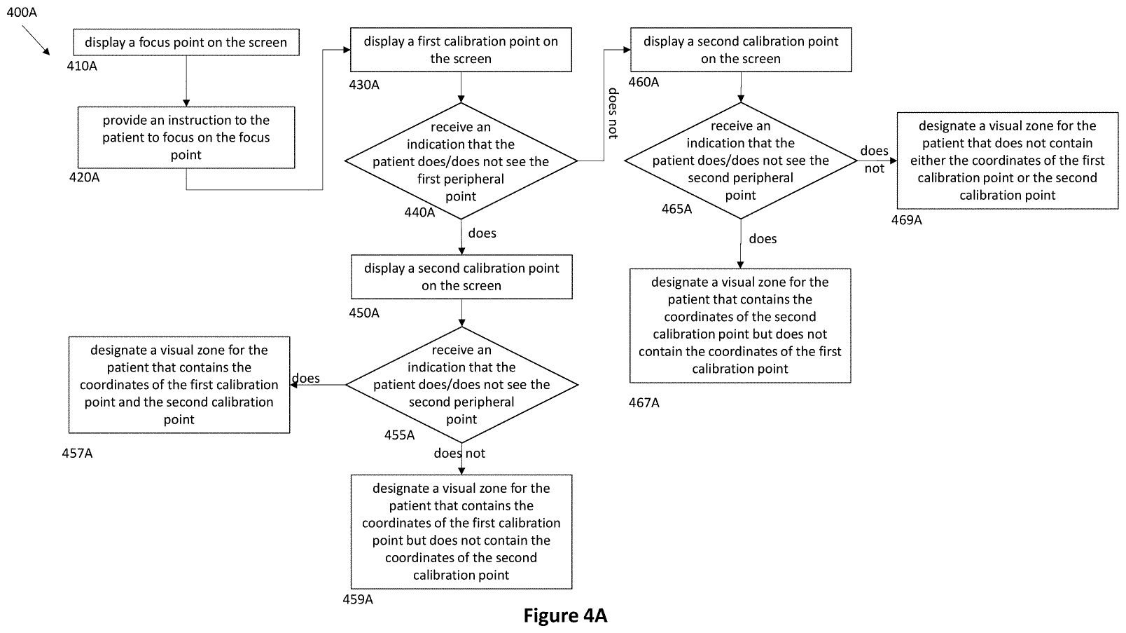

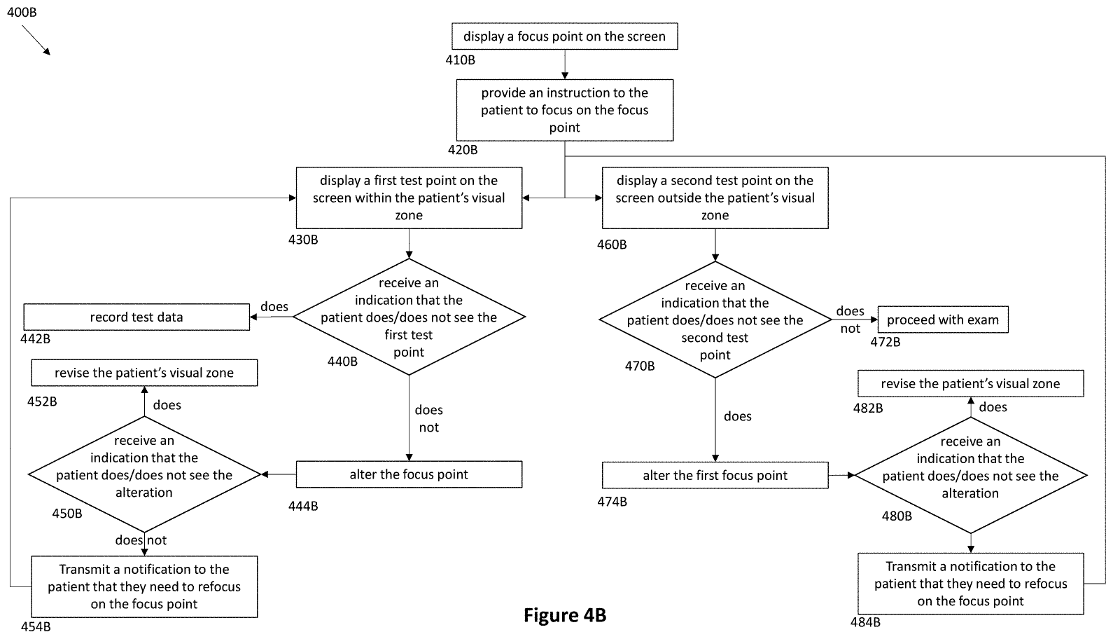

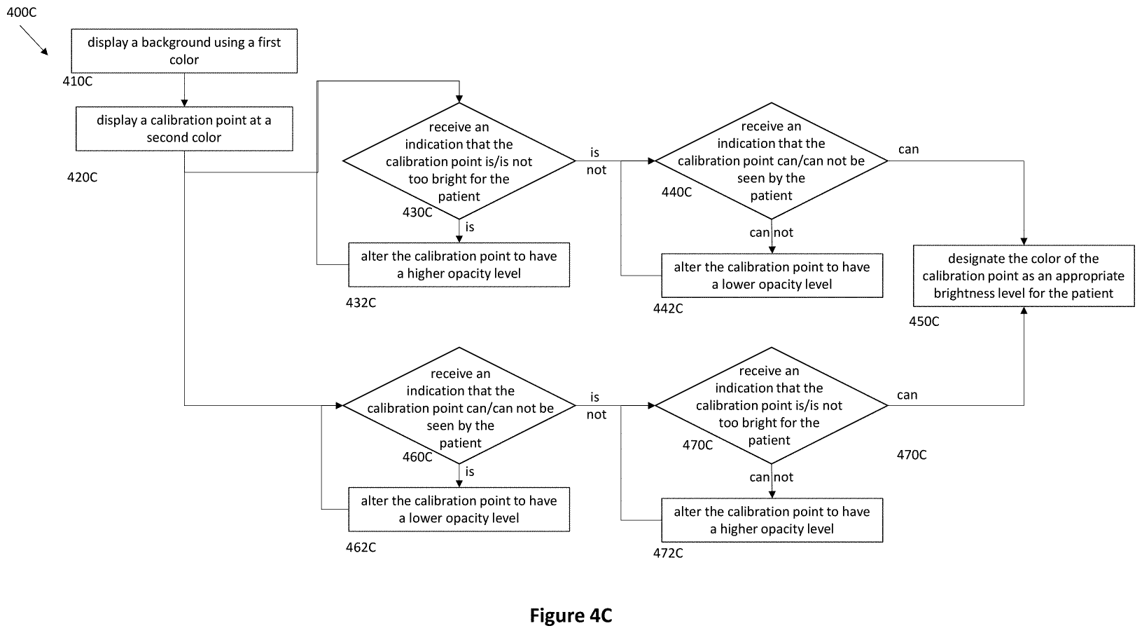

FIG. 4A shows an exemplary calibration method 400A for calibrating a visual zone of a patient. In Step 410A, the calibration system first displays a focus point on a screen of the headset, preferably at the center of the screen, and instructs the patient to focus on the focus point in step 420A. The instruction could be provided in an audio format or a visual format, or a combination of both (e.g., an instruction provided in an audio format via speakers in the headset, optionally using subtitles that are displayed within a designated portion of the screen). In some embodiments, the focus point may change over time to “gamify” the focus point test and ensure that the patient looks at the focus point. For example, the patient may be provided a bonus point in a tally that is provided in a score form to the user at the end of the test and an audio “beep” feedback if the patient actuates one user interface switch (e.g., by pulling one trigger) when the focus point changes to a certain letter or logo. Where the user fails to respond to the alteration of the focus point within a threshold period of time, the user could be docked points to produce a lower numeric “score,” or a tone could be transmitted to the user to indicate a failure threshold condition being detected.

After a focus point has been displayed on the screen, the calibration system displays a calibration point on the screen in step 430A and receives a signal from the user interface indicating whether the patient does or does not see the calibration point in step 440A. Such an indication could be received in any suitable form, for example by actuating a switch (by, e.g., pulling a trigger), or by receiving an audio signal. In some embodiments, an indication that the patient does not see a calibration point could be in the form of an absence of a signal. For example, where a patient is instructed to pull a trigger, e.g., a right trigger, when the patient sees a calibration point, the system could interpret a pulled trigger within an appropriate reaction time to be an indication that the patient sees the calibration point. By contrast, the absence of a pulled trigger within the patient's known reaction time will be interpreted as an indication that the patient does not see the calibration point.

When the system receives an indication that the patient sees a calibration point in step 450, the system could be configured to ensure that the calibration point is within the designated visual zone for that screen of that patient. When the system receives an indication that the patient does not see a calibration point in step 460A, the system could be configured to ensure that the calibration point is not within the designated visual zone for that screen of that patient. As the system gathers more calibration point data, the system could re-define the borders of the visual zone by displaying points just within and just outside the borders of the currently defined visual zone. For example, in step 450A, the system then displays a second calibration point after the system receives an indication that the user sees the first calibration point. If the system receives an indication that the user sees the second calibration point, then in step 457A the system designates a visual zone for the patient that contains the coordinates of the first calibration point and the second calibration point. If the system receives an indication that the user does not see the second calibration point, then in step 459A, the system designates a visual zone for the patient that contains the coordinates of the first calibration point but does not contain the coordinates of the second calibration point. In step 460A, the system displays a second calibration point after the system receives an indication that the user does not see the first calibration point. If the system receives an indication that the user sees the second calibration point, then in step 467A the system designates a visual zone for the patient that contains the coordinates of the second calibration point but does not contain the coordinates of the first calibration point. If the system receives an indication that the user does not see the second calibration point, then in step 459A, the system designates a visual zone for the patient that does not contain either the coordinates of the first calibration point or the second calibration point. Once the system has used some designated number (e.g., 10, 20, or 30) of calibration points to define a visual zone, the system could then be configured to display calibration points within, for example, 5 mm or 2 mm of the known visual zone borders to re-define the metes and bounds of the visual zone.

Such calibration methods could be implemented for each eye of a patient individually, or for both eyes of a patient simultaneously. In embodiments where the calibration method is implemented on one eye at a time, instructions provided by the system for the calibration method (where the instructions are visual instructions) and the focus points could be displayed on both screens at the same locations of both screens, but the calibration points could be displayed on only one screen to test the visual zone of that patient's eye. In embodiments where the calibration method is implemented on both eyes simultaneously, the instructions, focus points, and calibration points could be displayed on both screens at the same locations of both screens.

FIG. 4B shows an exemplary method 400B to verify that a patient's visual zone is still calibrated correctly, or to verify that a patient is still focusing on the focus point on the screen. In step 410B, the system displays a focus point on the screen. Displaying focus points on the screen are typical for conducting various tests performed on the patient, for example a strabismus measurement test. In step 420B, the system instructs the patient to focus on the focus point. In some embodiments the system could instruct the patient to focus on the focus point before displaying the focus point on the screen, while in others the instruction could be provided while displaying the focus point, or after displaying the first focus point. Such instructions could be provided in any suitable manner, for example via an audio instruction or a visual instruction by displaying instructive text on the screen.

The system could then conduct the test in step 430B by displaying a first test point on the screen. Such tests typically require some sort of feedback from the patient after the patient sees the first test point, for example by actuating a switch in the right-hand user interface 120, or by moving a user interface, which moves the test point on the screen. Such test feedback mechanisms are described in more detail below. The system detects whether the patient in step 440B sees the first test point by receiving such expected feedback, and if the system receives an indication that the patient sees the first test point, the system could then record test data as normal in step 442B.

However, a patient may indicate to the system that the patient does not see the first test point in step 440B. Such indications could be an absence of an expected triggering signal from a user interface, or they could be in the form of a signal from a user interface that the patient does not see the first test point. For example, if the patient does not see the first test point, the patient could actuate (e.g., pull the trigger of) the left-hand user interface 130 instead of the right-hand user interface 120, which indicates to the system that the patient does not see the first test point. In another embodiment, if the patient does not see the first test point in a threshold period of time, for example the patient's known maximum reaction time threshold, then the system could be programmed to understand that lack of response within the patient's known maximum reaction time threshold to be an indication that the patient does not see the first test point. At this point, the system could try to verify if the patient's visual zone has been compromised, or if the patient is not properly focused on the focus point displayed in step 410B.

In step 444B, the system could then alter the focus point to verify if the patient is still focused on the focus point. Such an alteration could be any suitable test, for example by changing a shape of the focus point from a circle to a square, or by changing the color, shade, or intensity of the focus point. Preferably, such alterations are subtle, such that they cannot be detected by a patient's peripheral vision, for example by shifting the opacity level of a color by less than 20% or by 10%, or by shifting the area of the shape of the focus point by no more than 20% or 10%. In step 450B, the system could then receive an indication of whether the patient sees the alteration to the focus point. For example, the patient could have been given an instruction before the exam that if the focus point changes in some manner, the patient should actuate the switch (e.g., pull the trigger) on the right-hand user interface 120 twice rapidly, or the patient should say “change,” which a microphone in the headset 110 receives. If the patient indicates that the patient does not see the alteration in the focus point, then in step 454B, the system could transmit a notification to the patient to refocus on the focus point, and it could then restart the test in step 430B.

If the patient indicates that the patient sees the alteration of the focus point in step 450B, then the system could register a flag that the patient's visual zone has changed since the previous calibration period. In some embodiments, the flag could trigger an initiation of a recalibration of the patient's visual zone. In other embodiments, the flag could trigger a notification to the patient that the patient's visual zone may have changed since the previous recalibration and could prompt the patient to take another visual zone recalibration test. In yet another embodiment, the flag could trigger a notification to a practitioner that the patient's visual zone may have changed. In some embodiments, the test could continue, and a notification or a recalibration test could only be triggered after a predetermined minimum number of flags have been registered or received by the system.

In some embodiments, during a test, the system could purposefully display test points outside the patient's visual zone to verify that the patient is still focused on the focus point. In step 460B, the system could display a second test point on the screen that is displayed outside the patient's known visual zone. In step 470B, the system receives an indication of whether the patient sees the second test point that is displayed outside the patient's known visual zone. If the system receives an indication that the patient does not see the second test point in step 470B, then the system could proceed with the exam in step 472B.

If the system receives an indication that the patient sees the second test point in step 470B, the system could, again, alter the focus point in step 444B to determine if the patient is still focused on the focus point displayed in step 410B, and could then await a response from the patient in step 480B. If the system receives an indication that the patient does not see the alteration of the focus point in step 480B, the system could then transmit a notification to the patient that they need to refocus on the focus point in step 484B, and the system could then continue with the exam. If the system receives an indication that the patient sees the alteration to the focus point in step 480B, the system could then, again, revise the patient's visual zone in step 482B in a similar manner as it revised the patient's visual zone in step 452B. In either case, the system has received an indication that the patient's visual zone may have changed since the previous calibration test.

As with the methods disclosed in FIG. 4A, such methods could be implemented for each eye of a patient individually, or for both eyes of a patient simultaneously. The methods could be implemented on only one screen, thereby testing one eye of the patient without needing to instruct the patient to close the other eye that is not being tested during a test.

FIG. 4C shows an exemplary method 400C to determine an appropriate virtual brightness for a patient. Since typical screens do not allow applications to alter the brightness of a screen, the brightness of an item that is displayed on a screen can be virtualized by altering an opacity of a color. For example, a bright yellow, red, or green color could have an opacity filter of 10%, 30%, or 50% placed on the bright color to make it appear to be less bright, even if the brightness on the screen remains the same. Such brightness tests could be used to alter the colors used by the system and verify that the colors can be easily seen by the patient. Preferably, such brightness tests are performed for multiple primary colors, for example red, green, and blue, as many tests are performed using different colors, and some patients may not see some colors as well as other colors.

In step 410C, the system displays a background shade, such as black, white, or grey, and in step 420C, the system displays a calibration point in a color that contrasts with the background shade, such as a red dot on a black background, or a green dot on a grey background. In step 430C, the system could query the patient to determine whether the calibration point is too bright for the patient. If the calibration point is too bright for the patient, then in step 432C, the system could alter the calibration point to have a higher opacity level, such as an opacity level of 30% instead of an opacity level of 20%. The system could then query the patient again in step 430C to determine if the calibration point is too bright until the patient indicates that the calibration point is not too bright.

The system then preferably verifies that the patient can still see the calibration point in step 440C. If the patient indicates that the calibration point cannot be seen, then in step 442C, the system lowers the opacity level of the calibration point, preferably to a level that is not lower than the last calibration point that was indicated to be too bright for the patient. For example, if the patient indicates that an opacity level of 20% is too bright, but an opacity level of 40% cannot be seen, then the system could set the opacity level to 30% for the next cycle. The system continues to verify that the calibration point can be seen in step 440C, and when an appropriate virtual brightness/opacity level has been set for that color, the system could then select that color as an appropriate brightness level for that patient in step 450C.

In some embodiments, the system may first wish to start with a high opacity color and decrease the opacity. In such embodiments, the system could prompt the patient to indicate whether the patient can see the calibration point, and it receives an indication in step 460C. If the system receives an indication that the patient cannot see the calibration point at the high opacity level, the system could then lower the opacity level in step 462 and then re-solicit input in step 460C. As before, after the system receives an indication that the patient can see the calibration point, the system could then solicit a response from the patient of whether the calibration point is too bright for the patient in step 470C. If the system receives an indication that the calibration point is too bright, the system could then alter the calibration point to have a higher opacity level in step 472C, preferably an opacity level that is not higher than an opacity level that was indicated to be not seen by the patient instep 460C, and it could then resolicit input in step 470C until the patient indicates that the calibration point is not too bright. Once the patient indicates that the opacity level is not too bright in step 470C, the system could designate the color at an appropriate brightness level for that color in step 450C.

In some embodiments, the system could perform tests to determine the upper and lower bounds of the patient's brightness tolerances, and it then could set the brightness level of the patient to have an opacity level that is between the patient's upper and lower opacity bounds. For example, the system could determine the lower bound of the patient's opacity level to be 20% and the upper bound to be 60%, and it could then choose 40% to be the most appropriate opacity level for the patient.

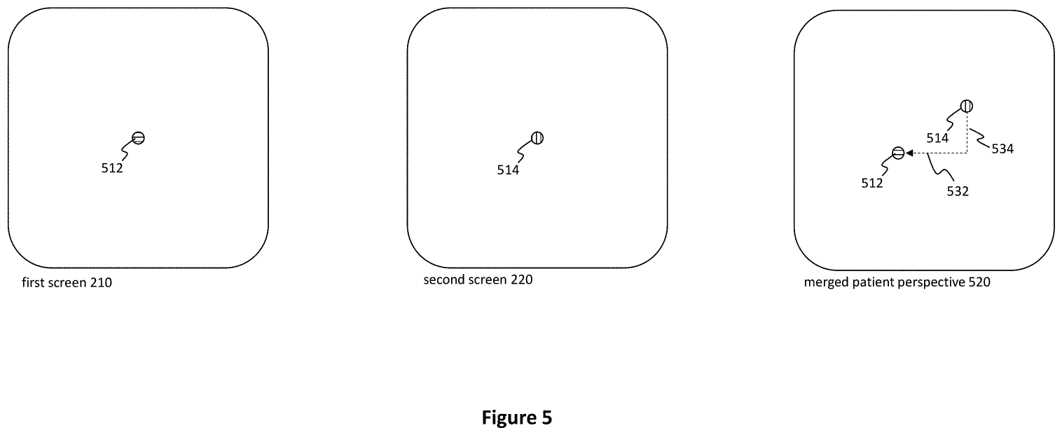

FIG. 5 shows a first display or screen 210 (e.g., a left screen) and a second display or screen 220 (e.g., a right screen) used to perform a strabismus measurement for a patient. In such an embodiment, the headset system could be configured to display a first test point 512 on the first display 210 and a second test point 514 on the second display 220 within the patient's visual zone. Preferably, the test points 512 and 514 are both displayed at the same coordinates for each of the displays 210 and 220, respectively. Here, the patient has a strabismus and sees an image 520, where the first test point 512 does not overlap or coincide with the second test point 514, despite the fact that both test points are displayed in the same coordinates for each of the displays 210 and 220. The system could then solicit feedback from the patient to determine what the patient sees. A patient who indicates to the system that the patient only sees one point may be recognized by the system to not have a strabismus issue, but a patient who indicates to the system that the patient sees two different points may be recognized by the system to have a strabismus issue.

Where the system recognizes the patient to have a strabismus issue, the system could then measure the severity of the strabismus by allowing the patient to use a user interface to move a displayed point from one location on a screen to another location, until, to the user, both points align with one another. For example, here, the patient may be indicated to move the first test point 512 to overlap or coincide with the second test point 514, and/or to move the second test point 514 to overlap or coincide with the first test point 512. The system could then measure the horizontal deviation 532 and the vertical deviation 534. The system could be configured to display the test points 512, 514 in different colors, such as red and green, or blue and yellow, to allow for easy differentiation between the points. The horizontal and vertical deviations could be measured and saved as test data to indicate the patient's strabismus severity, and the system could save historical test results to allow a patient or a practitioner to see how a strabismus condition may change over time.

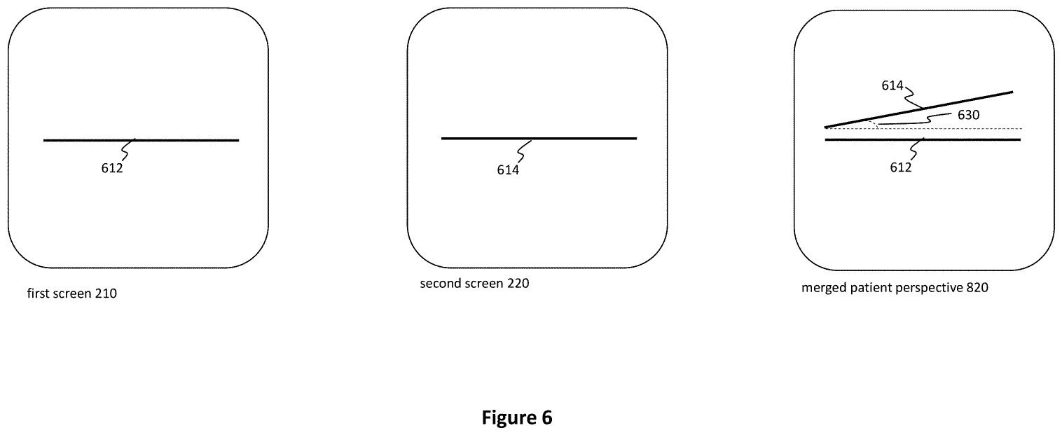

FIG. 6 shows the first (e.g., left) display or screen 210 and the second (e.g., right) display screen 220 configured for performing a torsion measurement for a patient. The headset system could be configured to display a first test line 612 on first display 210, and a second test line 614 on the second display 220. While the test lines 612 and 614 are shown here as horizontal lines, any suitable lines could be displayed, such as vertical lines or diagonal lines, in other embodiments. Preferably, the headset system is configured to ensure that the test lines 612, 614 are displayed within the known visual zones of the patient, and within the known luminance limits for the patient. The test lines 612 and 614 are preferably displayed in the same location for each screen having the same starting and ending coordinates and the same angle of rotation, such that a patient with perfect vision would see both lines overlapping or coinciding with one another. The system could then solicit feedback from the patient to determine what the patient sees. A patient who indicates to the system that the patient only sees one line may be recognized by the headset system to have perfect vision, but a patient who indicates to the system that the patient sees two different lines may be recognized by the headset system to have a torsion issue. A patient with a torsion problem will see an image 820, in which the first and second test lines do not coincide.

Patients that are recognized to have a torsion issue could have the severity of the torsion measured by allowing a user to move and rotate a line from one location to another until, to the user, both lines align with one another. For example, the patient could be instructed to move the first line 612 over the second line 614 until they overlap or coincide, and/or the patient could be instructed to move the second line 614 over the first line 612 until they overlap or coincide. Each line could be colored differently to allow for easy differentiation between the lines—for example the first line 612 could be red and the second line 614 could be blue. The patient rotates at least one of the lines 612, 614 through an angle of rotation 630 to align the lines 612 and 614 with one another in the image 820, and the angle or rotation 630 can be measured and saved to calculate the patient's torsion severity. The system could save historical test results to allow a patient or a practitioner to see how a torsion may change over time.

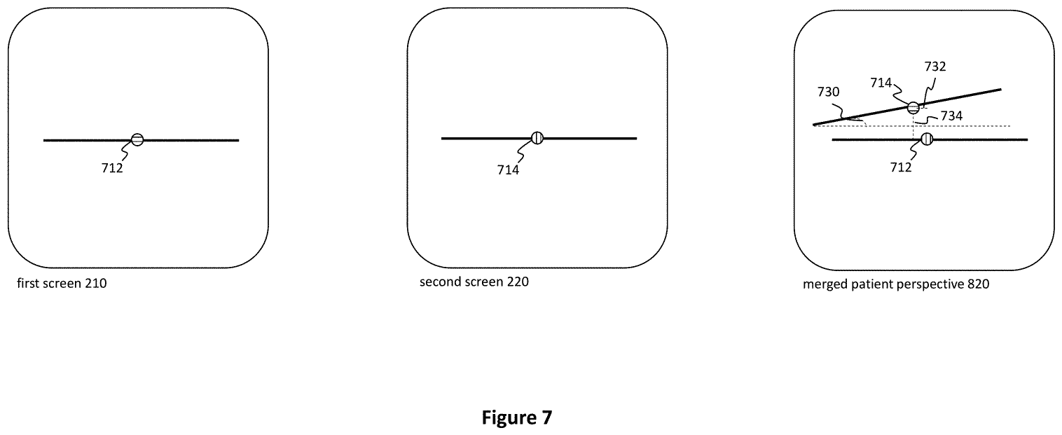

FIG. 7 shows the first display or screen 210 and the second display or screen 220 configured to perform a combined strabismus and torsion measurement for a patient. In this embodiment, a first line 712 is displayed on the first display 210, and a second line 714 is displayed on the display 220. The first line 712 has a first enlarged central point 713, and the second line 714 has a second enlarged central point 715. Here, the patient has both a strabismus problem and a torsion problem, and a single test could be used to measure the horizontal deviation, vertical deviation, and angle of rotation for each of these deviations. Accordingly, when the patient moves the second line 714 to overlap or coincide with the first line 712, the central points 713, 715 will necessarily overlap or coincide, thereby providing an image 820′ including a horizontal deviation measurement 732 and a vertical deviation measurement 734 in addition to an angled deviation measurement 730. While the lines with enlarged central points are shown and used here as exemplary, any suitable shape could be used, such as that of an animal, which provides a level of whimsy and fun to the test.

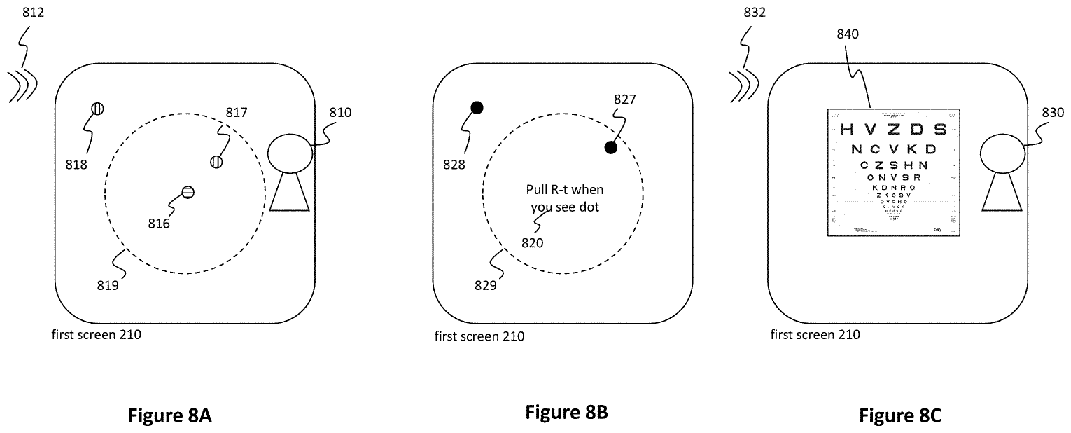

FIGS. 8A-8C show a first screen or display 210 configured with different embodiments of a “virtual assistant” to assist a patient when interacting with a test. In FIG. 8A, the first screen 210 has a visual virtual assistant 810 and an audio virtual assistant 812 configured to provide instructions to a patient when conducting a calibration test to determine a patient's blind spot reactions. The visual virtual assistant 810 is shown here as a visual representation of doctor that is guiding a patient through the calibration test, while the audio virtual assistant 812 is shown as sounds emanating from a speaker (not shown) of a headset system, such as the headset 110. In preferred embodiments, the visual virtual assistant 810 and the audio virtual assistant 812 are pre-recorded using 3-D recording equipment to provide a rendering of a three-dimensional visual and audio representation of an eye care practitioner that is saved to a memory. The headset system could then be configured to render appropriate pre-recordings via the screens and speakers of a headset system.

In some embodiments, the instructions provided by the virtual assistant could be configured to be sequential instructions, such as an instruction to look at a focus point 816, and actuate a switch (e.g., a trigger) on a user interface, such as right-hand user interface 120, when a first dot or point 817 is seen within a visual field 819 while the patient is looking at the focus point 816. In preferred embodiments, the instructions provided by the virtual assistant could be configured to be selected in response to feedback received from a patient. For example, if the headset system receives a signal indicating that a patient sees a second dot or point 818 displayed outside of the patient's known visual field 819 (e.g., a switch, such as a trigger, is actuated after the second dot 818 is displayed on the first screen 210), the headset system could provide an instruction to the patient to focus on the focus point 816. The headset system could also provide an instruction to the patient to actuate a switch (e.g., pull a trigger) when the focus point 816 is altered, such as if it changes to a different color or shakes or rotates in place. If the headset system triggers the focus point 816 to change to a different color, but it does not detect the designated switch actuation, then the headset system could also provide a reminder to the patient via the virtual assistant to focus on the focus point 816 in a suitable manner, for example, by having the audio virtual assistant 812 tell the patient to focus on the focus point 816 while the visual virtual assistant 810 points at the focus point 816.

FIG. 8B shows an alternative visual virtual assistant 816 which comprises instructions that act as the focus point for the patient. In this embodiment, the patient is encouraged to look at instructions 820 while dots or points 827 within the patient's visual field 829 and dots or points 828 outside the patient's visual field 829 are displayed on the first screen 210. Such an alternative visual virtual assistant 816 could also comprise warnings, and feedback (positive and/or negative) in response to input received from a user interface of the headset system. FIG. 8C shows yet another alternative visual virtual assistant 830 and an audio virtual assistant 832 that provide feedback instructions for a patient to interact with a screen 840 that shows testing data. By providing a screen 840 upon which a patient may view testing data, the patient is further immersed within the augmented reality presented via the headset system. Such a screen 840 could be displayed on a wall of a virtual office, or in any other suitable setting that further immerses a patient within a setting such that the patient feels as if engaging with an actual practitioner.

It will be appreciated from the foregoing that the headset visual test systems and methods disclosed herein can be adapted to a wide variety of uses systems, and that systems employing the disclosed features can be operated to calibrate and perform visual tests for a patient as will be suitable to different applications and circumstances. It will therefore be readily understood that the specific embodiments and aspects of this disclosure described herein are exemplary only and not limiting, and that a number of variations and modifications will suggest themselves to those skilled in the pertinent arts without departing from the spirit and scope of the disclosure.

What is claimed is:

1. A system for conducting vision tests on a patient, comprising:
a headset configured to be worn by the patient;
a display in the headset configured to be visible to the patient when the headset is worn by the patient;
a user interface configured to receive a physical or verbal input from the patient and to transmit a signal in response to the physical or verbal input;
a memory having stored therein a set of software instructions; and
a processor configured, when the headset is worn by the patient, to execute software instructions in the set of software instructions that cause the processor to:
provide a focus point on the display in a visual zone of the display in which the patient can focus on the focus point;
provide a peripheral point on the display within a designated blind spot zone for the patient;
receive the signal from the user interface when the user interface receives a physical or verbal input from the patient indicating that the patient sees the peripheral point; and
transmit a notification to the patient when the signal from the user interface indicates that the patient is not focused on the focus point.

2. The system of claim 1, wherein the display comprises:
a first display configured to be visible to one eye of the patient when the headset is worn by the patient, and a second display configured to be visible to the other eye of the patient when the headset is worn by the patient;
wherein the computer processor is further configured, when the headset is worn by the patient, to:
provide a second focus point on the second display in a position in which the patient can focus on the second focus point;
provide a second peripheral point on the second display within a second designated blind spot zone for the patient;
receive a signal from the user interface indicating that the patient sees the second peripheral point; and
transmit a notification to the patient when the user interface indicates that the patient is not focused on the second focus point.

3. The system of claim 2, wherein the first focus point and the second focus point are provided in different coordinates on the first and second displays, respectively.

4. The system of claim 1, wherein the processor is further configured, when the headset is worn by the patient, to execute software instructions in the set of instructions that cause the processor to:
provide a first calibration test point on the display;
receive a first signal from the user interface that the patient sees the first calibration test point on the display;
record a first time delay between the transmission of the first calibration test point and the reception of the first signal;
provide a second calibration test point on the display;
receive a second signal from the user interface indicating that the patient sees the second calibration test point on the display;
record a second time delay between the transmission of the second calibration test point and the reception of the second signal; and
generate a maximum reflex time that is greater than both the first time delay and the second time delay.

5. The system of claim 4, wherein the processor is further configured, when the headset is worn by the patient, to execute instructions in the set of instructions that cause the processor to:
provide a first diagnostic test point on the display;
monitor inputs from the user interface to determine whether the patient sees the first diagnostic test point on the display; and
provide a second diagnostic test point on the display after at least the maximum reflex time has passed since the first diagnostic test point has been provided.

6. The system of claim 1, wherein the display is a first display, the system further comprising a second display configured to be positioned in front of the other eye of the patient, wherein the processor is further configured to execute instructions in the set of instructions that cause the processor to:
provide a first test line within the visual zone of the patient on the first display;
provide a second test line within the visual zone of the patient on the second display;
provide an instruction to the patient to move the second test line to visually align with the first test line;
receive an input from the user interface to move the second test line on the second display to a torsion measurement configuration; and
measure a rotation of the torsion measurement configuration to calculate a torsion deviation of an eye of the patient.

7. The system of claim 6, wherein the processor is further configured to execute instructions in the set of instructions to:
provide an instruction to the patient to move the first test line to visually align with the first test line;
receive an input from the user interface to move the first test line to second torsion measurement configuration; and
measure a rotation of the second torsion measurement configuration to calculate a torsion deviation of the other eye of the patient.

8. The system of claim 7, wherein the processor is further configured to execute instructions in the set of instructions to provide the first calibration line in a first color and the second calibration line in a second color different from the first color.

9. The system of claim 1, wherein the display is a first display configured to be visible to one eye of the patient, the system further comprising a second display configured to be visible to the other eye of the patient, wherein the computer processor is further configured to execute instructions from the set of instructions to:
provide a first test point on the first display within a first visual zone for the patient;
provide a second test point on the second display within a second visual zone for the patient;
provide an instruction to the patient to move the second test point to visually align with the first test point;
receive an input from the user interface to move the second test point to a strabismus measurement configuration; and
measure a horizontal deviation and a vertical deviation of the strabismus measurement configuration to calculate a strabismus deviation of at least one eye of the patient.

10. A method of conducting vision tests using a headset worn by a patient, the headset having a first display positioned to be visible to one eye of the patient and a second display positioned to be visible to the other eye of the patient, the method comprising:
displaying a first focus point on the first display;
providing an instruction to the patient to focus on the first focus point;
displaying a first calibration point on the first display;
displaying a second calibration point on the first display, wherein the first focus point, the first calibration point, and the second calibration point each have different coordinates on the first display;
receiving a signal from a user interface indicating that the patient sees the first calibration point and does not see the second calibration point; and
generating a first visual zone for the patient that contains the coordinates of the first calibration point but does not contain the coordinates of the second calibration point.

11. The method of claim 10, further comprising:
displaying a test point on the first display outside the generated visual zone for the patient;
receiving a signal from the user interface that indicates that the patient sees the test point; and
transmitting a notification to the patient that the patient is not focused on the first focus point.

12. The method of claim 10, further comprising:
transmitting a first test point to display on the first display;
receiving a first signal from the user interface indicating that the patient sees the first test point on the first display;
recording a first time delay between the transmission of the first test point and the reception of the first signal;
transmitting a second test point to display on the first display;
receiving a second signal from the user interface indicating that the patient sees the second test point on the first display;
recording a second time delay between the transmission of the second test point and the reception of the second signal; and
generating a maximum reflex time that is greater than both the first time delay and the second time delay.

13. The method of claim 12, further comprising:
displaying a third test point on the first display;
monitoring inputs from the user interface to determine whether the patient sees the third test point on the first display;
waiting for at least the maximum reflex time before displaying a fourth test point on the first display; and
recording inputs from the user interface to determine whether the patient sees the fourth test point on the first display.

14. The method of claim 13, wherein the third test point and the fourth test point are transmitted to display within the generated first visual zone for the patient.

15. The method of claim 10, further comprising:
displaying a second focus point on the second display;
providing an instruction to the patient to focus on the second focus point;
displaying a third calibration point on the second display;
displaying a fourth calibration point on the second display, wherein the second focus point, the third calibration point, and the fourth calibration point each have different coordinates on the second display;
receiving an input from the user interface indicating that the patient sees the third calibration point and does not see the fourth calibration point; and
generating a second visual zone for the patient on the second display that contains the coordinates of the third calibration point but does not contain the coordinates of the fourth calibration point.

16. The method of claim 15, further comprising:
displaying a first test line on the first display within the first visual zone for the patient on the first screen;
displaying a second test line on the second display within the second visual zone for the patient on the second display;
providing an instruction to the patient to move the second test line to visually align with the first test line;
receiving an input from the user interface indicating that the second test line has been moved to a torsion measurement configuration; and
measuring a rotation of the torsion measurement configuration to calculate a torsion deviation of one eye of the patient.

17. The method of claim 16, further comprising:
providing an instruction to the patient to move the first test line to visually align with the second test line;
receiving an input from the user interface indicating that the first test line has been moved to a second torsion measurement configuration; and
measuring a rotation of the second torsion measurement configuration to calculate a torsion deviation of the other eye of the patient.

18. The method of claim 16, wherein the step of displaying the first calibration line on the first display comprises displaying the first calibration line on the first display in a first color and the step of displaying the second calibration line on the second display comprises displaying the second calibration line on the second display in a second color different from the first color.

19. The method of claim 10, further comprising:
displaying a first test point on the first display within the first visual zone for the patient on the first display;
displaying a second test point on the second display within a second visual zone for the patient on the second display;
providing an instruction to the patient to move the second test point to visually align with the first test point;
receiving an input from the user interface indicating that second test point has been moved to a strabismus measurement configuration; and
measuring a horizontal deviation and a vertical deviation of the strabismus measurement configuration to calculate a strabismus deviation of an eye of the patient.
